(19) United States
(12) Patent Application Publication
(10) Pub. No.: US 2023/0256166 A1
(43) Pub. Date: Aug. 17, 2023

(54) PORTABLE INFUSION PUMP WITH REMOTE ACTIVATOR

(71) Applicant: Modular Medical, Inc., San Diego, CA (US)

(72) Inventors: Paul M. DiPerna, San Diego, CA (US); Marc D. Goldman, San Diego, CA (US); Kelsie DiPerna, San Diego, CA (US)

(21) Appl. No.: 17/673,415

(22) Filed: Feb. 16, 2022

(51) Int. Cl. A61M 5/172; G16H 40/67; G16H 20/17

(52) U.S. Cl. CPC A61M 5/172; G16H 40/67; G16H 20/17; A61M 2205/3553; A61M 2205/505; A61M 2205/3389; A61M 2205/581; A61M 2205/582; A61M 2205/587; A61M 2205/3317; A61M 2205/3327; A61M 2205/609; A61M 2205/80

(57) ABSTRACT

A system for activating a portable infusion pump includes a remote interaction device operated by the user for direct communication with the pump. Upon receipt of an initiate signal from the user, a computer on-board the infusion pump evaluates status metrics from the infusion pump, along with clinical information, to validate the initiate signal. With this validation, the computer identifies an appropriate response for operating the infusion pump. An indicator on the infusion pump then informs the user of the appropriate response for the user to follow for activating the infusion pump.

### FIELD OF THE INVENTION

The present invention pertains to systems and methods which provide a user with the remote capability of requesting a specific operation of an infusion pump, without requiring tactile contact with the pump. More particularly, the present invention pertains to a computer application that is incorporated into an infusion pump for this purpose. The invention is particularly, but not exclusively, useful as a computer application that responds to voice activated instructions from a user for operating an infusion pump in compliance with a predetermined, clinically prescribed protocol.

### BACKGROUND OF THE INVENTION

A major concern for the administration of prescription medications is that the medication must be properly used in accordance with clinical and regulatory requirements. For long term treatment protocols, such as insulin injections, the administration of the protocol typically involves user participation. It is not uncommon, however, that a third-party caregiver becomes involved. If so, their involvement can complicate matters in several ways. This, of course, is to be avoided.

By way of example, consider an insulin delivery protocol wherein it may be desirable, or necessary, to deviate from a same delivery schedule. Specifically, this will typically be the case when there are activity changes in the user's daily routine. The situation becomes even more problematical when the user is incapacitated or is otherwise incapable of self-administering a desired change in the delivery protocol. In these scenarios, the reliance on a medically uncertified caregiver to administer a change in the delivery protocol may be unwise, and possibly illegal.

To overcome the difficulties and inconveniences note above, it is necessary for the user to somehow maintain his/her personal responsibility for affecting changes in a drug delivery protocol. Thus, when a user is physically unable to affect changes in a drug delivery protocol, the only option may be to do so verbally with a voice-activated insulin delivery pump.

For the reasons set forth above, it is an object of the present invention to provide a system and method for verbally instructing a computerized pump with an initiate message to execute specific changes in a preprogrammed medication delivery protocol. A further object of the present invention is to provide the computerized pump with the capability of analyzing the initiate message and providing the user with an indication of how to properly proceed. Still another object of the present invention is to have the user electronically verify the initiate message, with the consequence that the pump is either activated to perform the change order, or the pump informs the user there can be no activation due to operational/protocol noncompliance. Yet another object of the present invention is to provide a system and method for verbally instructing a computerized pump to execute an initiate message for a preprogrammed medication delivery protocol that is simple to use, easy to manufacture and comparatively cost effective.

### SUMMARY OF THE INVENTION

A system for activating an infusion pump includes a remote interaction device which is operated by a user to activate the infusion pump. The system also includes a computer which is mounted on the infusion pump and is connected for two-way communication with the interaction device. Typically, the computer is pre-programmed with a protocol that is determined in accordance with the prescription of a licensed clinical person, namely a physician.

An activating switch is provided on the remote interaction device for sending an initiate message from the user to the computer. In this initiate message, the user states his/her intentions for activating the infusion pump. Normally these intentions refer to temporary deviations from the pre-programmed protocol in the computer. Preferably, the initiate message is voice activated.

Upon its receipt of the initiate message, the computer validates the message. This validation is based on a computer evaluation of the operational status metrics compiled by the computer for the infusion pump. More specifically, the evaluation is made by analyzing requirements presented in the initiate message, with consideration given to the operational capabilities of the infusion pump identified by the status metrics, and clinical considerations established by the protocol.

For an operation of the present invention, an indicator is mounted on the infusion pump and connected with the computer. The purpose of the indicator is to identify an appropriate action to be followed by the user for an operation of the infusion pump. Specifically, this appropriate action is based on the computer evaluation, and it is manifested by the computer as an indicating signal to the user. As envisioned for the present invention, the indicating signal can be either audible tones, vibrations, and lights, or a combination of these.

Upon receipt of an indicating signal, the user can respond in any of several ways. Thus, a response button for activating the infusion pump can be operated in several modes. For one, the user can respond via a voice communication between the interactive device and the infusion pump. On the other hand, a physical response button can be provided on the infusion pump for operation by the user. In any event, the response action will typically include a choice between i) activating the pump in accordance with the initiate message, revising the initiate message, and iii) accepting a default with no further action. When a tactile response is to be made, a physical response button on the infusion pump may be either a capacitive button, an accelerometer to register taps and shakes, or a biometric reader.

A methodology for an operation of the present invention requires three essential components. One is a portable interaction device which can be operated by a user to activate an infusion pump. Another is the infusion pump itself. And a computer which is mounted on the infusion pump and is preprogrammed for infusing a fluid medicament from the infusion pump to the user. To implement this methodology, a two-way communication system is provided between the interaction device and the infusion pump. As envisioned for the present invention, this communication between the user and the computer on the infusion pump will typically proceed according to the following scenario.

First, the computer will receive an initiate message from the user wherein the user presents his/her intentions for activating the infusion pump. Upon receipt of the initiate message, the computer evaluates the operational status metrics that are pertinent to an operation of an infusion pump. This evaluation is then used by the computer for validating an operation of the infusion pump. Further, validation is accomplished by evaluating the status metrics of the infusion pump with consideration given to the user's intentions expressed in the initiate message. An indicating signal is then presented at the infusion pump which will support a response signal by the user for an appropriate action from the infusion pump.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of operational components for the present invention; and

FIG. 2 is a functional flow sequence of the interactive steps involved in an operation of the present invention.

### DESCRIPTION OF THE PREFERRED EMBODIMENTS

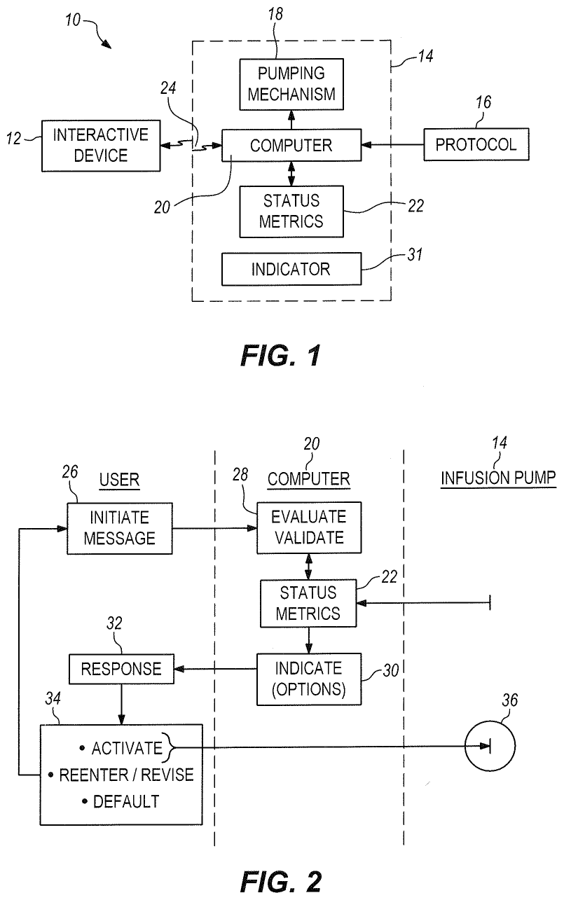

Referring initially to FIG. 1 a system for infusing a fluid (liquid) medicament to a user is shown and is generally designated 10. As shown, the system 10 includes an interactive device 12 that is directly operated by the user (not shown). FIG. 1 also shows an infusion pump 14 is included in the system 10, and that the infusion pump 14 will normally be automatically operated in accordance with a pre-programmed protocol 16. As envisioned for the present invention, the protocol 16 will typically follow a specified operational regimen for an infusion therapy, and it will typically be prescribed by a physician.

With reference to FIG. 1, it is to be appreciated that the infusion pump 14 includes a pumping mechanism 18 which is under the control of a computer 20. As a component of the system 10, the computer 20 is physically mounted on the infusion pump 14. FIG. 1 also shows that the computer 20 has an interactive connection with status metrics 22 that are pertinent to the operation of the infusion pump 14. Specifically, the status metrics 22 will typically include information on the operational condition of the infusion pump 14, as well as parameters for compliance with the protocol 16. FIG. 1 further shows that communication between the computer 20 and the interactive device 12 is provided via a two-way communications link 24. Preferably, the two-way communications link 24 is voice activated, although other modes of communications, such as a “texting” operation, are possible for an operation of the present invention.

The sole function of the interactive device 12 is to initiate an activation of the infusion pump 14. As shown in FIG. 2, this initiation is accomplished by sending an initiate message 26 from the user to the computer 20. The initiate message 26 does not however, by itself, activate the infusion pump 14. Instead, the initiate message 26 expresses the intentions of the user and it will typically include both temporal and quantitative information for a desired operation of the infusion pump 14. Importantly, although the time, duration and quantity of an infusion may be changed by the initiate message 26, the present invention requires the initiate message 26 be checked and verified before the infusion pump 14 is activated.

In FIG. 2 it is seen that when an initiate message 26 is sent to the computer 20, it is immediately checked and verified by an evaluate/validate function 28 of the computer 20. In detail, the evaluate aspect of function 28 is accomplished, in part, with reference to the status metrics 22 that have been compiled by the computer 20. This evaluation of the status metrics 22 is also based on reference to the protocol 16 via its connection with the computer 20 as shown in FIG. 1. The validate aspect of the function 28 is thus accomplished based on both operational and clinical information, with consideration given to the user's intentions expressed in the initiate message 26. Moreover, the validate aspect of the function 28 creates an indicating signal 30 that appears on an indicator 31 which is mounted on the infusion pump 14, as shown in FIG. 1. Specifically, the indicator 31 presents a response option 32 that may either support or reject the intentions presented in the initiate message 26.

Once the initiate message 26 has been evaluated and validated the indicating signal 30 can be created on the indicator 31 in any of several modes. For instance, the indicating signal 30 may be audible tones, vibrations, lights, or a combination thereof.

The response option 32 provided by the indicating signal 30 will typically include a choice between several courses of action for the user; see block 34 of FIG. 2. These options include: i) activating an operation 36 of the infusion pump 14 in accordance with the initiate message 26, ii) revising the initiate message 26 for compliance purposes, and iii) accepting a default with no further action. As indicated above, the particular response option 32 which is selected by the user can be manifested either verbally using the interactive device 12, or tactilely. When a tactile response is to be made, a response button (not shown) on the infusion pump 14 may be used. As envisioned for the present invention, this can be either a capacitive button, an accelerometer to register taps and shakes, or a biometric reader.

While the particular Portable Infusion Pump with Remote Activator as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for activating an infusion pump which comprises:
a remote interaction device operated by the user of the infusion pump;
a computer mounted on the infusion pump and connected therewith, wherein the computer includes a communication unit for two-way communication with the interaction device, and a validation unit for evaluating operational status metrics compiled for the infusion pump;
an activating switch for sending an initiate message from the user to the communication unit of the computer, wherein the initiate message includes the user's intentions for activating the infusion pump;
an indicator mounted on the infusion pump to inform the user of an appropriate response action to be taken by the user, wherein the indicating signal is activated by the computer in response to the initiate message, and is based on a validated computer evaluation of the status metrics; and
a response button for activation of the infusion pump by the user when activation is an appropriate response.

2. The system of claim 1 wherein the response action is selected from the group consisting of i) pump activation in accordance with the initiate message, ii) await a revision to the initiate message, and iii) default with no further action.

3. The system of claim 1 wherein the computer is pre-programmed for infusing a fluid medicament to the user in accordance with a predetermined protocol, and wherein the computer maintains status metrics regarding protocol compliance.

4. The system of claim 3 wherein the protocol is established by a licensed medical professional.

5. The system of claim 3 wherein the status metrics include medication volume, time interval between medication dosages, and infusion rate.

6. The system of claim 1 wherein the indicating signal is selected from the group consisting of audible tones, vibrations, and lights.

7. The system of claim 1 wherein the response button is selected from the group consisting of a capacitive button, an accelerometer to register taps and shakes, a biometric reader, and a voice response from the user.

8. The system of claim 1 wherein the computer reacts to a default by requiring a response action selected from the group consisting of:
creating a new initiate order; and
sending a noncompliance signal to a predetermined clinical entity for recordation and possible inquiry.

9. The system of claim 8 wherein the clinical entity is the user's primary physician.

10. The system of claim 1 wherein the activating switch is voice-activated.

11. A method for remotely activating an infusion pump which comprises the steps of:
providing a portable interaction device for operation by a user;
mounting a computer on the infusion pump, wherein the computer is preprogrammed for infusing a fluid medicament from the infusion pump to the user;
transferring status metrics pertinent to an operation of the infusion pump to the computer for use in validating an operation of the infusion pump;
incorporating a communication unit with the computer for two-way communication with the interaction device;
sending an initiate message from the user to the computer, wherein the initiate message indicates the user's intentions for activating the infusion pump;
evaluating the status metrics of the infusion pump to validate the initiate message; and
creating an indicating signal at the infusion pump, wherein the indicating signal is activated by the computer in response to the initiate message and is based on a validated computer evaluation of the status metrics of the infusion pump for supporting a response signal by the user for an appropriate action by the infusion pump.

12. The method of claim 11 wherein the indicating signal identifies a step from the group consisting of:
activating the infusion pump for an operation in accordance with the initiate message;
revising/reentering the initiate message; and
taking no further action due to an operational noncompliance.

13. The method of claim 11 wherein the status metrics include medication volume, time interval between medication dosages and infusion rate.

14. The method of claim 11 wherein the indicating signal is selected from the group consisting of audible tones, vibrations, and lights.

15. The method of claim 11 wherein the response signal is selected from the group consisting of a capacitive button, an accelerometer to register taps and shakes, a biometric reader, and a voice response from the user.

16. The method of claim 11 wherein the initiate message is voice activated.

17. A non-transitory, computer readable medium having executable instructions stored thereon that direct a computer to perform a process, the medium comprising instructions for:
receiving an initiate message from the user to the computer, wherein the initiate message indicates the user's intentions for activating the infusion pump;
transferring status metrics pertinent to an operation of an infusion pump to the computer for use in validating an operation of the infusion pump;
evaluating the status metrics of the infusion pump to validate the initiate message; and
creating an indicating signal at the infusion pump, wherein the indicating signal is activated by the computer in response to the initiate message, and is based on a validated computer evaluation of the status metrics of the infusion pump for supporting a response signal by the user for an appropriate action by the infusion pump.

18. The medium of claim 17 wherein the status metrics include medication volume, time interval between medication dosages and infusion rate.

19. The medium of claim 17 wherein the indicating signal is selected from the group consisting of audible tones, vibrations, and lights.

20. The medium of claim 17 wherein the response signal is selected from the group consisting of a capacitive button, an accelerometer to register taps and shakes, a biometric reader, and a voice response from the user.
